(19) United States
(12) Patent Application Publication
(10) Pub. No.: US 2022/0259611 A1
(43) Pub. Date: Aug. 18, 2022

(54) METHOD FOR INCREASING YIELD IN PLANTS

(71) Applicants: LIMAGRAIN EUROPE, Saint-Beauzire (FR); INSTITUT NATIONAL DE RECHERCHE POUR L'AGRICULTURE, L' ALIMENTATION ET, L'ENVIRONNEMENT, Paris (FR); Centre National de la Recherche Scientifique, Paris (FR)

(72) Inventors: Bertrand HIREL, Versailles Cedex (FR); Hervé LASSAGNE, Chappes (FR); Sébastien PRAUD, Glaine Montaigut (FR); Marie-Héléne TIXIER, Chappes (FR); Thérèse TERCÉ-LAFORGUE, Saint- Germain-en-Laye (FR)

(21) Appl. No.: 17/624,754

(22) PCT Filed: Jul. 3, 2020
(86) PCT No.: PCT/EP2020/068825

§ 371 Date: Jan. 4, 2022

(30) Foreign Application Priority Data
Jul. 5, 2019 (EP) 19305924.3

(51) Int. Cl. C12N 15/82; A01H 6/46

(52) U.S. Cl. CPC C12N 15/8261; A01H 6/4684

(57) ABSTRACT

The invention relates to a method for obtaining plants presenting a higher yield by inhibition of the GDH2 activity in the plants.

### FIELD AND BACKGROUND OF THE INVENTION

The present invention relates to the field of improving grain yield for plants, in particular improving grain yield in cereals and in particular improving grain yield in maize under optimal nitrogen (N) fertilization conditions. This is particularly obtained by down regulating NAD-dependent glutamate dehydrogenase 2 (GDH2) activity, in particular by inhibiting the expression thereof. The invention thus comprises such methods and plants presenting such a down-regulation.

The cereals including maize, wheat and rice account for 70% of worldwide food production. When such crops are grown for seed protein content and biomass production, they require large quantities of nitrogenous fertilizers to attain maximal yields. In the past few years, there has been considerable interest in nitrogen use efficiency (NUE), which can be defined as the kernel or biomass yield per unit of nitrogen (N) in the soil and the N utilization efficiency (NutE), which is the yield per N taken up (Hirel et al., J. Exp. Bot. 58: 2369-2387, 2007). There is an increasing interest this day to optimize plant use of nitrogen in order to limit the use of nitrogenous fertilizers by farmers.

Maize (Zea mays L.), also called corn, is now ranked first among cereals, comprising 41% of the total world cereal production. A doubling of maize production has occurred over the last 30 years, with almost 1,000 million metric tons (38,105 bushels) being produced in 2015-2016 (https://corn3blog.wordpress.com/global-comparison/). With yields of over 10 metric tons per ha, maize also ranks first in terms of grain yield both in Europe and in the USA, although in the rest of the world, the yield is much lower, accounting for approximately 5-6 metric tons per ha (http://www.agprofessional.com/news/A-comparison-of-world-corn-yields-227415201. html).

The main findings concerning both the physiological function and the putative role of GDH in the control of plant growth and development are described below.

In most plant species GDH is encoded by two distinct nuclear genes (Melo-Oliveira et al., Proc. Natl. Acad, Sci. USA 96: 4718-4723, 1996; Pavesi et al., Genome 4: 306-316 2000; Restivo, Plant Sci. 166: 971-982, 2004). Each gene encodes a different subunit termed α- and β-polypeptides, which can be assembled as homo or heterohexamers composed of different ratio of α- and β-polypeptides thus leading to the formation of seven active isoenzymes. These seven isoenzymes can be distinguished using native polyacrylamide gel electrophoresis followed by in-gel NAD-dependent activity (Turano et al., Plant Physiol. 112: 1357-1364, 1996) or NADH-dependent activity staining (Loulakakis and Roubelakis-Angelakis, Physiol. Plant. 96: 29-35, 1996). Variations of the GDH isoenzymes pattern were observed according to both the organ examined and the N source (Loulakakis and Roubelakis-Angelakis, Planta 187: 322-327,1992; Turano et al., Plant Physiol. 113:1329-1341,1997; Fontaine et al., Plant Cell Physiol. 47: 410-418, 2006).

The occurrence of a third active GDH subunit (termed γ) has been demonstrated in the roots of Arabidopsis thaliana. The ability of the γ-subunit to assemble with the α- and β-subunits (Fontaine et al., Plant Signal. Behay. 8: 3, e233291-5, 2013) has rekindled an interest in investigating more closely the metabolic or regulatory role of the different GDH subunits.

However, the physiological significance of the organ- species- and metabolic-dependent variability of the ratio between the two or the three GDH isoenzyme subunits and its regulation is still unclear and may not solely be explained in terms of metabolic function (Skopelitis et al., Plant Cell 18: 2767-278, 2006; Purnell et al., Planta 222: 167-180, 2005; Fontaine et al., Plant Signal. Behay. 8: 3, e233291-5, 2013). As in the vast majority of the other plant species examined so far, only two distinct genes encoding α and β GDH subunits have been identified in maize (Hirel at al. Physiol. Plant. 124: 167-177, 2005). This finding suggests that in maize the α- and β-GDH subunits GDH have a species-specific metabolic or regulatory function in regulating the C/N balance of the plant which is likely to be different to that found in Arabidiopsis (Fontaine et al., Plant Cell 24: 4044-4065, 2012).

Several studies have evaluated GDH for improving maize productivity.

In maize plants overexpressing the E. coli gene gdhA, which encodes NADPH-GDH, the kernel biomass was higher than the controls when the plants were grown in the field under drought conditions (Lightfoot et al., Euphytica 156: 103-116, 2007). This was in line with the finding that in maize, QTLs for GDH activity colocalized with QTLs for kernel yield (Dubois et al., Plant Physiol Biochem. 41:

565-576, 2003) and that in rice overexpressing a fungal GDH, grain yield was increased (Zhou et al., Crop Sci, 55: 811-820, 2015).

U.S. Pat. No. 5,879,941 describes the use of several plant species, including maize, transformed with nucleotide sequences encoding the α- and β-subunits of the Chlorella sorokiniana NADPH-GDH. These plants exhibited improved properties such as increased growth and enhanced osmotic stress tolerance. In tobacco, it has been shown that the additional GDH activity, was able to divert plant metabolism when the α and β subunits were overexpressed either individually or simultaneously (Tercé-Laforgue et al., Plant. Cell. Physiol. 54: 1634-1647, 2013), thus rendering the transgenic plants more resistant to salt stress (Tercé-Laforgue et al., Plant Cell Physiol. 56: 1918-1929, 2015).

In WO2011036160, the GDH2 enzyme from maize is exemplified amongst other sequences. However, this application doesn't provide any working example with this sequence. Instead, it contains examples pertaining to overexpression of GDH1 in rice, which led to an increased seed yield.

In WO2014164014, the GDH2 from sorgho is exemplified. This document is dealing mainly with silencing but doesn't describe any experiments and doesn't provide any results with this sequence. In particular, this document doesn't provide any information as to which trait could be modified by such silencing.

U.S. Pat. Nos. 7,485,771 and 5,998,700 present results about an overexpression of the GDHA (another name for GDH2) gene.

Fontaine et al. (2012) describe a gdh2 Arabidopsis mutant and study the role of the GDH enzyme.

Fontaine et al. (2006) disclose the down-regulation of GDHA in tobacco with the antisense strategy, and don't present any data pertaining to yield.

In Grabowska et al. (2017), the GDH2 from triticale was both overexpressed and downregulated (antisense construct) in Arabidopsis thaliana. This publication is merely analyzing the activity of GDH2 and they do not present any result related to the improvement of plant agronomic performances.

In its thesis “Nitrate: Metabolism and Development”, Castro Marin described attempts to obtain homozygous Arabidopsis thaliana mutants null for GDH2. The author states in particular that no homozygous T-DNA insertion plants were identified from the screening of GDH2 and GDH3 plants, and that, in RNAi plants, the activity appeared to be around 48% decreased. Furthermore, the author didn't observe significant changes in the transgenic plants compared to the WT. These results are in contradiction with the ones reported in the present examples, where lack of GDH2 activity is reported, presence of homozygous plants following transposon insertion has been verified, and increase in amino acid content has been observed. Without being bound by this theory, the difference between the results of Castro Marin and the data reported herein may be due to the nature of the plant development where Arabidopsis thaliana, a dicotyledonous plant may have a further need than the monocotyledonous plants that are cereals, in particular maize, which have a different development.

Mara et al (Microb Cell Fact., 2018 Nov 1;17(1):170) describe the pleiotropic effects of the Glutamate Dehydrogenase (GDH) pathway in Saccharomyces cerevisiae, and that the oxidizing form of GDH (NAD-GDH) activity is encoded by GDH2 gene in yeast. They further report that gdh2Δ cells presented wild type growth and did not display any deficiencies due to glutamate homeostasis impairment.

In summary, none of the patents or studies listed above, alone or in combination, disclose nor suggest that there is a link between the downregulation of GDH2 and yield improvement in optimal conditions in particular in cereals.

Unexpectedly, the inventors have demonstrated that it is possible to increase yield in cereals, by inhibiting (down-regulating) GDH2 gene expression in these plants.

### DESCRIPTION OF THE INVENTION

In summary, the invention relates to a cereal comprising at least one cell (preferably all cells) which presents an inhibition of the GDH2 activity.

In a specific embodiment, inhibition of the GDH2 activity is due to a mutation in the gdh2 gene.

The cereal of the invention is a cereal wherein the mutation in the gdh2 gene is obtained by one of the following methods:
a. A physical treatment
b. A chemical treatment, or
c. An engineering biological system

A physical treatment can be application of an electromagnetic radiation, such as gamma rays, X rays, and UV light, or of a particle radiation, such as fast and thermal neutrons, beta and alpha particles.

A chemical treatment can be treatment of seeds, gametes or plant parts with EMS (Ethyl Methanesulfonate) or sodium azide.

An engineering biological system can be Gene editing, base editing or Genetic Modification (GM).

The cereal of the invention is a cereal plant wherein the mutation in the gdh2 gene is not obtained by an essentially biological process.

In another embodiment, inhibition of the GHD2 activity is due to the presence in the genome of said cereal, of an antisense, or of an overexpression construct (leading to co-suppression), or of an RNAi construct. These constructs are engineering biological system.

In a preferred embodiment, the gdh2 gene encodes the GDH2 enzyme depicted by SEQ ID NO: 1 (GDH2 of maize) or an orthologue thereof in another plant, said orthologue of the GDH2 enzyme presenting at least 86% identity with SEQ ID NO: 1.

In a preferred embodiment, the cereal is maize.

In a preferred embodiment, inhibition of the GDH2 activity is due to an insertion between nucleotides 814 and 815 of SEQ ID NO: 2, in particular an insertion of a transposon between such nucleotides.

In a specific embodiment, inhibition of the GHD2 activity is due to introduction of a mutation in SEQ ID NO: 2, by deletion of all or part of SEQ ID NO: 2, by introduction of mutations or deletion in the promoter (in the 100 bp that are 5′ of SEQ ID NO: 2 in the plant gene), being performed by gene editing.

The invention also relates to a method for improving yield in a cereal, comprising inhibiting the GDH2 activity in said cereal, wherein said cereal with the inhibited GDH2 activity presents a higher yield than a cereal not having an inhibited GDH2 activity.

The invention also relates to a method for producing a cereal with improved yield, comprising the step of inhibiting the GDH2 activity in said cereal, in particular maize, inhibition which can be obtained by:

a. An insertional mutagenesis or the introduction of at least one point mutation within the gdh2 gene.

b. The expression of an antisense or RNAi construct or the overexpression of a sense construct to cause co-suppression wherein the constructs are integrated in the cereal's genome, or

c. the removal of part of the gdh2 gene or the entire gene by gene editing

The invention also relates to a method for increasing cereal yield, comprising the step of sowing cereal seeds, wherein said cereal seeds grow into plants that exhibits an inhibited GDH2 activity, and wherein the yield of the harvested cereals is increased as compared to the yield obtained from harvested cereals not exhibiting an inhibited GDH2 activity.

The invention also relates to a method for selecting a cereal with improved yield comprising the step of selecting, in a population of cereals, the cereals in which the GDH2 activity is inhibited.

The invention also relates to a method for identifying a cereal of the invention comprising the steps of: (a) screening a population of cereals, and (b) identifying the cereals presenting an inhibition of the GDH2 activity.

The invention thus pertains to a cereal that contains at least one cell which presents total or partial inhibition of the expression of a gene coding for the GDH2 protein as described above. Preferably, the cereal presents more than one cell having said inhibition, and in particular all cells of the cereal present said inhibition. This is in particular the case when the inhibition is due to the presence of a “determinant”, i.e. a modification that has been introduced within the cell genome by a man-driven manipulation. Such cereal will present a higher yield than a cereal in which said inhibition is not present, in normal conditions. Interestingly, the inventors were able to show that the effect was observed with homozygous or heterozygous plants.

In the context of the present invention yield is the amount of seeds harvested from a given acreage. It can thus be expressed as the weight of seeds per unit area. It is often expressed in metric quintals (1 q =100 kg) per hectare in the case of cereals. It can also be defined as seed yield.

A cereal of the invention can be chosen amongst the following species: maize, wheat, rice, sorgho, barley, millet. The cereal is preferably maize.

### Ortholoques

The sequence listing provides examples of GDH2 genes or proteins for various cereal species. In particular, the sequence for the maize enzyme is provided as SEQ ID NO: 1. Sequences for sorghum, rice, barley and wheat are provided as SEQ ID NO: 3 to SEQ ID NO: 8 respectively (three sequences for the three wheat genomes). The coding genes are provided as SEQ ID NO: 2 (maize) and SEQ ID NO: 9 to SEQ ID NO: 14 for sorghum, rice, barley and wheat.

It is clear that these sequences represent sequences of some alleles of the genes, and that the invention can also be performed in plants in which the gene sequence corresponds to another allele. It is also possible to identify orthologues of such genes in other species than the one herein disclosed.

According to the invention, a GDH2 orthologue is a protein presenting at least 86% of sequence identity with SEQ ID NO: 1 (Accession NP_001132187.1), preferably at least 88% sequence identity, preferably at least 90% sequence identity, preferably at least 95% sequence identity, preferably at least 98% sequence identity, preferably at least 99% sequence identity. In a preferred embodiment, the GDH2 orthologs are chosen amongst sequences SEQ ID NO: 3 to SEQ ID NO: 8.

The degree of identity between a target sequence and a sequence of reference is determined by comparison of the target sequence with the sequence of reference over the whole length of the sequence of reference.

Essentially, the “percentage of sequence identity” can be determined by comparing two optimally aligned sequences over a comparison window, where the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. It is however preferred when the percentage of identity is obtained by using the BLASTP algorithm (Altschul et al, (1997), Nucleic Acids Res. 25:3389-3402; Altschul et al, (2005) FEBS J. 272:5101-5109), using the default algorithm parameters, and in particular the scoring parameters:
Matrix: BLOSUM62
Gap Costs: Existence: 11; Extension: 1
Compositional adjustments: Conditional compositional score matrix adjustments.

### Inhibition of the GDH2 Activity

According to the invention, the inhibition of the GDH2 activity can be achieved by different methods, which are preferably man-driven. This means that the element that will lead to the inhibition of the GHD2 activity will be introduced in the plant with human activity at one stage at least.

Inhibition can be achieved at the genomic level, in particular by introducing a mutation or insertion in the promoter or in the gene, or by removal of part of the gdh2 gene or the entire gene.

Inhibition can be achieved at the transcriptional level, meaning that there is no gdh2 transcript or less gdh2 transcripts or shorter gdh2 transcripts.

Inhibition of activity can be achieved at the protein level, by producing less GDH2 protein, a truncated GDH2 protein or even no GDH2 protein.

In a specific embodiment, said gdh2 gene expression is inhibited in multiple cells of said cereal, wherein said inhibition in multiple cells results in an inhibition in one or several tissues of said cereal. In this embodiment, it is possible that the gdh2 gene is not inhibited in other tissues of said cereal.

In another embodiment, said gdh2 gene expression is inhibited in all cells of said cereal. In this case, the invention encompasses a cereal which presents an inhibition of the expression of the gdh2 gene.

It is to be noted that the invention envisages that the inhibition of gdh2 gene expression is obtained by various methods, such as mutation of the gene or transformation of the plant with a vector that will eventually cause the inhibition. In particular, the inhibition is obtained by the introduction of a “determinant” in cells of said cereal. As foreseen herein, a “determinant” causes the inhibition of gdh2 gene, is inheritable from generation to generation and is transmissible to other plants through crosses. Determinants will be described in more details below and include mutations and transgenes (introduced foreign DNA within the genome of the cells of the plant).

### Total or Partial Inhibition

As foreseen in the present invention, a total inhibition of a gene coding the GDH2 protein in a cell indicates either that:
(i) No gdh2 mRNA is detected in said cell after RNA isolation and reverse transcription or Northern Blot. In particular, total inhibition is obtained when no mRNA is detected after RNA isolation and reverse transcription of such. This may be obtained by mutating the gdh2 gene, in particular by introducing a missense mutation near the transcription starting site or by impairing the sequence of the gdh2 promoter. The skilled person knows how to identify the essential features like the TATA box or the CAAT box in a promoter and mutate it in order to impair its functionality.
(ii) No functional protein is produced in said cell. No functional protein is produced in absence of gdh2 mRNA (see (i)). In other cases, mRNA may be present but leads to the production of a truncated protein (as an illustration when the gdh2 mRNA is incomplete, in particular in case of the presence of a mutation within the gene, in an intron or in an exon). Truncated proteins can be detected by isolation of the proteins, Western Blot and detection of the size of the protein with an antibody (polyclonal or monoclonal) directed against the GDH2 protein. Presence of mutations (substitution of amino-acids) within the protein may also lead to production of a non-functional protein.

As foreseen in the present invention, a partial inhibition of a gene coding for the GDH2 protein in a cell indicates gdh2 mRNA is detected in said cell after RNA isolation and reverse transcription or Northern Blot, but at a lower level than that detected in a cell which do not bear the determinant introduced within the cell to induce gdh2 inhibition. In particular, partial inhibition is obtained when a lower level of mRNA is detected after RNA isolation and reverse transcription. In particular, partial inhibition is obtained when the level of gdh2 mRNA is lower than 0.9 times, more preferably lower than 0.75 times, and more preferably lower than 0.66 times of the level of gdh2 mRNA in a cell which does not bear the element (determinant) leading to gdh2 inhibition. Preferably, the control cell that is used to make the comparison is from a plant that is isogenic to the plant from which originates the cell in which partial inhibition is to be detected. Preferably, the cells are from the same plant tissue and mRNA is isolated at the same level of development. It is indeed most preferred that the level of inhibition is compared from comparable cells, only differing from the presence or absence of the element inducing inhibition.

The level of gdh2 mRNA can be measured as an absolute level. It is nevertheless preferred that the level of gdh2 mRNA is measured as a relative level, compared to other control genes. In this case the method to be used to measure the level of mRNA and to detect inhibition is as follows:
mRNA is isolated from tissues in which it is supposed to be inhibited and control tissues (such as leaves for example)
Reverse transcription and real-time quantitative PCR are performed on said mRNA using primers that amplify the gdh2 gene or primers that amplify control genes. These control genes are genes which are known to be usable as control in Northern Blot analysis, as their quantity level rarely varies. One can cite actin, ubiquitin 2, EF1α genes. It is preferred that at least two control genes are used, and in particular ubiquitin 2 and EF1α.
The Cp is then calculated for each amplified sample according to methods known in the art for real-time qPCR. In particular, machine used to perform real-time qPCR usually present a software which can automatically calculate this value, by calculation of the second derivative maximum.
One then calculates the value equal to 2exp-(Cp for gdh2 mRNA—Cp for control gene (or mean of Cp of the control genes). This value gives a relative level of expression for gdh2 mRNA as compared to the level of expression of the control gene. If this value is higher than 1, this means that there is more gdh2 mRNA than the control gene. If this value is lower than 1, this means that there is less gdh2 mRNA than the control gene.
One then compares the values obtained for the cells in which gdh2 is inhibited (i.e. where the determinant is present) and for the cells in which gdh2 is at the basal level (i.e. where the determinant is absent). Ratio between these two values allows the determination of the level of inhibition of the gdh2 gene. Such partial inhibition can be obtained from a heterozygous plant (only one copy of the genes is inhibited) or with RNAi (as there may be some leakage).

### Mutation of the gdh2 Gene

In an embodiment, inhibition of the GDH2 activity is obtained by a mutation of the gdh2 gene through insertional mutagenesis or the introduction of at least one point mutation. In particular, the mutations are introduced by the person skilled in the art in the genome of the plant of the invention and are thus man-made.

In this embodiment, expression and/or activity of GDH2 is inhibited by mutagenesis of the gene coding for said protein.

The mutagenesis of the gene can take place at the level of the coding sequence or of the regulatory sequences for expression, in particular of the promoter. It is, for example, possible to delete all or part of said gene or promoter and/or to insert an exogenous sequence.

By way of example, mention will be made of insertional mutagenesis: a large number of individuals derived from a cereal that is active in terms of the transposition of a transposable element (such as the AC or Mutator elements in maize) are produced, and the cereals in which there has been an insertion in the gdh2 gene are selected, for example by PCR.

It is also possible to introduce at least one point mutation with a physical treatment (electromagnetic radiation, such as gamma rays, X rays, and UV light, and particle radiation, such as fast and thermal neutrons, beta and alpha particles.) or a chemical treatment, such as EMS or sodium azide treatment of seed or by using a biological engineering system such as gene editing, base editing or Genetic Modification (GM). The consequences of these mutations may be to shift the reading frame and/or to introduce a stop codon into the sequence and/or to modify the level of transcription and/or of translation of the gene. In this context, use may in particular be made of techniques of the “TILLING” type (Targeting Induced Local Lesions IN Genomes; McCALLUM et al., Plant Physiol., 123, 439-442, 2000). Such mutated cereals are then screened, in particular by PCR, using primers located in the target gene. One can also use other screening methods, such as Southern Blots or the AIMS method that is described in WO 99/27085 (this method makes it possible to screen for insertion), by using probes that are specific of the target genes, or through methods detecting point mutations or small insertions/deletions by the use of specific endonucleases (such as Cel I, Endo I, which are described in WO 2006/010646).

In this embodiment, the determinant as mentioned above is the mutation (transposon or point mutation(s)) that is introduced in the genome. It is indeed inheritable and transmissible by crosses.

In another embodiment, inhibition of the GDH2 activity is due to the presence in cells of said cereal of an antisense construct, or of an overexpression construct (that will lead to co-suppression of the gene), or of a RNAi construct. The DNA constructs used in these methods are introduced in the genome of said cereal through methods known in the art.

The transformation of cereal cells can be achieved by any one of the techniques known to one skilled in the art.

In particular, it is possible to cite methods of direct transfer of genes such as direct micro-injection into plant embryos, vacuum infiltration or electroporation, direct precipitation by means of PEG or the bombardment by gun of particles covered with the plasmidic DNA of interest.

It is preferred to transform the cereal with a bacterial strain, in particular Agrobacterium, in particular Agrobacterium tumefaciens. In particular, it is possible to use the method described by Ishida et al. (Nature Biotechnology, 14, 745-750, 1996) for the transformation of Monocotyledons.

In particular, inhibition may be obtained by transforming the cereal with a vector containing a sense or antisense construct. These two methods (co-suppression and antisense method) are well known in the art to permit inhibition of the target gene. One can also use the RNA interference (RNAi) method, which is particularly efficient for extinction of genes in plants (Helliwell and Waterhouse, 2003). This method is well known by the person skilled in the art and comprises transformation of the cereal with a construct producing, after transcription, a double-stranded duplex RNA, one of the strands of which being complementary of the mRNA of the target gene.

In another embodiment, inhibition of the gdh2 activity is due to an engineering biological system such as gene editing tools. In particular, inhibition may be obtained by the removal of part of the gdh2 gene or the entire gene, the interruption of the endogenous promoter, the introduction of mutations or of a frameshift in the endogenous gene.

Such genome editing tool includes without limitation targeted sequence modification provided by double-strand break technologies such as, but not limited to, meganucleases, ZFNs, TALENs (WO2011072246) or CRISPR/CAS system (including CRISPR Cas9, W02013181440), Cpf1 (W02016205711) or their next generations based on double-strand break technologies using engineered nucleases. The CRISPR-associated nucleases can also be linked to a deaminase domain to induce a specific nucleotide replacement at a specific position by base editing.

The invention also relates to a method for producing a cereal with improved yield, comprising the step of inhibiting the GDH2 activity in said cereal.

In a particular embodiment, the step of inhibiting the GDH2 activity in said cereal is achieved by insertional mutagenesis in the gdh2 gene or in the promoter of the gene. The skilled person knows how to identify the essential features like the TATA box or the CAAT box in a promoter and mutate it in order to impair its functionality.

In another embodiment, the step of inhibiting the GDH2 activity is achieved by physical or chemical treatment that will induce at least one point mutation in the gdh2 gene or in the promoter, using the tools disclosed above.

In another embodiment, the step of inhibiting the GDH2 activity in said cereal is achieved by transformation of a vector comprising a RNAi construct or an antisense construct or a construct coding for GDH2 (co-suppression). In this embodiment, the cell contains the determinant (antisense, sense of RNAi construct, with the appropriate sequence and promoter) within its genome.

In another embodiment, the step of inhibiting the GDH2 activity in said cereal is achieved by an engineering biological system such as genome editing tools. This would thus include the steps of transforming the cells (or plants) with appropriate vectors for both expressing the nucleases, the guide(s) in the case of CRISPR-associated nuclease and optionally the template(s) needed for replacing the gene of the plant. The nuclease and the guide(s) may also be delivered directly into the cell as ribonucleoprotein complexes.

The invention relates to a method for identifying a cereal comprising at least one cell which presents an inhibition of the GDH2 activity comprising the step of screening a population of plants and identifying the desired plants. This method is performed in vitro, using molecular biology tools known in the art. One can cite, performing Western blots (to detect the presence of the protein), Southern blots (to detect and analyze the nature of the DNA of the plant, and detect deletion of all or part of the gene, presence of mutations . . . ) or Northern blots (to detect and analyze the presence and amount of gdh2 mRNA).

Means for performing the identification step above in such a method can be selected in the group consisting of:
at least two primers for amplifying a nucleic acid sequence of the invention,
at least one marker hybridizing to a nucleic acid sequence of the invention, and
at least one antibody recognizing the polypeptide of the invention.

In a particular embodiment, the primers can be chosen amongst the following sequences: SEQ ID NO: 22, SEQ ID NO: 23, SEQ ID NO: 24.

The invention also relates to a method for identifying, in a population of plants, a cereal comprising at least one cell which presents an inhibition of the GDH2 activity which comprises the step of identifying, in such population of plants, the plants containing the expression cassette (RNAi, antisense or sense for cosuppression), the mutation in the gdh2 gene, the presence or amount of gdh2 mRNA and/or the presence or amount of the GDH2 protein in a sample of the plants.

Such method is thus an in vitro method, intended to identify, in a population of plants, the ones that carry the transgene, mutation or determinant according to the invention. Identification/detection of the plants carrying such element is performed by using adequate samples from the various plants of the population.

In a specific embodiment, the identification is performed through the use of a marker (such as a probe) that is specific to the transgene. In this embodiment, the identification step is thus preferably preceded by a step comprising genotyping said population of plants.

In a specific embodiment, the identification step is preceded by a step comprising extracting the RNA from the individuals in said population.

In a specific embodiment, the identification step is preceded by a step comprising extracting the DNA from the individuals in said population.

In a specific embodiment, the identification step is preceded by a step comprising extracting proteins from the individuals in said population.

The inhibition of GDH2 activity can be detected by comparing/sequencing all or part of the genomes of the plants from the population and identifying mutations in the gdh2 gene or promoter. The inhibition of GDH2 activity can be detected by comparing the level of gdh2 transcripts in the plants from the population compared to controls. The inhibition of GDH2 activity can be detected by comparing the level of GDH2 protein in the plants from the population compared to controls.

The invention also relates to various methods of using the cereals of the invention.

In particular, the invention encompasses a method for improving yield in a cereal, comprising inhibiting the expression of a gene coding for the GDH2 protein as specified above in said cereal. In this method, the cereal with the inhibited gdh2 gene presents a higher yield than a second cereal not having an inhibited gdh2 gene, after sowing and harvest. In this method, it is clear that the increase of yield can be verified by sowing and harvesting of a multiplicity of cereals that present inhibition of the gdh2 gene, the yield of which is then compared with the yield obtained with a second group of cereals not presenting said inhibition, and this under the same culture conditions (sowing and harvest at the same time, on comparable field plots, use of the same amount of fertilizers, water . . . ). It is also preferred that comparison is to be performed on a second group of cereals that is isogenic to the cereals having the inhibited gdh2 gene. This “isogenic” cereal differs from the cereal having an inhibited gdh2 gene at very few loci (less than 20, more preferably less than 10), and does not carry the determinant leading to inhibition of the gdh2 gene (said determinant being the mutation in the gdh2 gene (coding DNA or regulatory sequences) or the construct leading to inhibition of expression of the gene or protein). This cereal can also be called “virtually isogenic”.

The invention also relates to a method for producing a cereal, comprising the step of inhibiting the expression of a gene coding for the GDH2 protein in said cereal. The inhibition of the gdh2 gene can be performed by any method as described above. Such cereal can later and optionally be used as in a breeding process for obtaining a cereal with improved yield. Indeed, it is often preferable to use, at a particular culture location, cereals lines that have been optimized for such location. Consequently, one can perform the genetic modifications (mutations, introduction of foreign DNA) in order to obtain a material that is then used for breeding process. The lines to be cultured are then obtained by introgressing the determinant leading to inhibition of the gdh2 gene in specific lines having otherwise agronomic quality characteristics optimized for the intended purpose. The introgression of the characteristic is in particular carried out by selection, according to methods known in the art (crossing and self-pollination). The plants are in particular selected using molecular markers, indicating the presence or absence of traits of interest.

A series of back crosses can be performed between the elite line (in which one wishes to introduce the determinant) and a line that already carries said determinant (the donor line). During the back crosses, one can select individuals carrying the determinant and having recombined the smallest fragment from the donor line around the determinant. Specifically, by virtue of molecular markers, the individuals having, for the markers closest to the determinant, the genotype of the elite line are selected. In addition, it is also possible to accelerate the return to the elite parent by virtue of the molecular markers distributed over the entire genome. At each back cross, the individuals having the most fragments derived from the recurrent elite parent will be chosen.

The invention thus also encompasses a method for selecting/identifying a cereal comprising the step of a) selecting, in a population of cereals, the cereals in which the gdh2 gene is inhibited. Such method is thus performed in vitro, by generic molecular genetic techniques (use of molecular markers, PCR, arrays and the like). Said selected cereal is suitable to be later used and can later be used in a breeding process for obtaining a cereal with improved yield.

In a specific embodiment, said population of cereals, in which the selection/identification is performed, is the progeny obtained from a cross between a first cereal line in which the gdh2 gene is inhibited and a second cereal line in which the gdh2 gene is not inhibited. Said inhibition of the gdh2 gene is caused by the presence of a determinant (mutation or foreign DNA as described above) within the genome of the cereal. Said selection/identification in step a) is thus performed by identification, in the genome of said cereal, of the presence of said determinant causing gdh2 inhibition (either directly through analysis of the genomic DNA of the cereal, or indirectly through analysis of the presence or absence of the products that should be obtained from the gdh2 gene (mRNA from the gdh2 gene and/or presence or absence of a functional or truncated GDH2 protein).

In a specific embodiment, step a) is performed through the use of a marker that is specific to the determinant leading to the inhibition of said gdh2 gene. In this embodiment, step a) is thus preferably preceded by a step comprising genotyping said population of cereals. In another embodiment, step a) is preceded by a step comprising extracting the RNA from the individuals in said population and performing a Northern Blot (or an equivalent method) in order to identify the cereals in which the production of mRNA from the gdh2 gene is inhibited. RNA may be extracted from specific tissues only. In another embodiment, the selection in step a) is preceded by a step comprising extracting proteins from the individuals in said population and performing a Western Blot (or an equivalent method) in order to identify the cereals in which the production of GDH2 protein is inhibited. Protein may be extracted from some specific tissues only.

Means for performing the identification step above in such a method can be selected in the group consisting of:
at least two primers for amplifying a nucleic acid sequence of the invention,
at least one marker hybridizing to a nucleic acid sequence of the invention, and
at least one antibody recognizing the polypeptide of the invention.

In a particular embodiment, the primers can be chosen amongst the following sequences: SEQ ID NO: 22, SEQ ID NO: 23, SEQ ID NO: 24.

The invention also comprises a method for obtaining a cereal exhibiting increased yield, comprising the step of introgressing the determinant responsible for the inhibition of the gdh2 gene into said cereal.

This method comprises the steps consisting in
a) crossing a first cereal line that has said determinant with a second cereal that does not have said determinant,
b) selecting, in the progeny obtained, the cereals in which the gdh2 gene is inhibited and which present the best genome ratio as regards said second cereal
c) performing a back cross of said selected cereals with said second cereal line
d) repeating, if necessary, steps b) and c) until an isogenic line of said second cereal, and containing said determinant, is obtained,
e) optionally, performing a self-pollination in order to obtain a cereal that is homozygous for said determinant.

The invention also relates to a method for increasing a cereal yield for a cereal harvest, comprising the step of sowing cereal seeds, wherein said cereal seeds grow into plants that exhibits an inhibited expression of a gdh2 gene, and wherein the yield of the harvested cereals is increased as compared to the yield obtained from harvested isogenic cereals which do not exhibit said inhibited expression of gdh2 gene. If necessary, the comparison may be performed as mentioned above. The invention also relates to a method of growing cereals, comprising the step of sowing the cereals of the invention, and growing cereals from the sowed seeds. The invention may also comprise the step of harvesting said cereals. The invention also relates to a method for harvesting cereals comprising the step of harvesting cereals of the invention.

It is clear that the cereals used in the methods described below are cereals according to the invention, in which the gene gdh2 (or the production of the GDH2 protein) is inhibited, either totally or partially, in all cells or in specific tissues, totally in some tissues whereas not at all or only partially in other tissues, or with the same level of inhibition in all tissues, as described above. It is also clear that all teachings and embodiments are applicable to a cereal which presents inhibition of the expression of the gdh1 gene (the other gene encoding GDH) in addition to the inhibition of gdh2 gene expression.

### DESCRIPTION OF THE FIGURES

FIG. 1: Structure of the 5′ region of the GDH2 gene (Zm00001d025984), position of the insertion site D0425 of the transposable element and position of the three primers used

FIG. 2: In gel GDH activity in L1 WT, L1 homozygous mutants, and L1 heterozygous mutants

FIG. 3: One-way ANOVA graphs of Grain Yield (GY15) (A) and Kernel numbers per square meters (K/m²) (B) by hybrids alleles type for optimal condition. Means diamonds correspond to 95% confidence intervals for each mean. On the right-hand side of the graph, a Dunnett's test was used to test for differences between the wt control and the other groups. The selected mean (wt as a control: in grey and bold) has a bold grey circle. Means that are significantly different from the selected mean have black bold circles and the corresponding groups are in black end bold (mm and m+).

FIG. 4: Amino acid content in roots of maize hybrids in which the mutation for the gene encoding GDH2 has been introduced. Wild type (WT), mm (homozygous mutation), m+ (heterozygous mutation). Results are the mean of four individual plants±Standard Deviation.

FIG. 5: Distribution of the plants according to the KASP analysis. Homozygous mutants upper left cluster, dashed line; heterozygous mutants, middle cluster, semi-dashed line; homozygous wild-type, bottom right cluster, plain line.

### EXAMPLES

### Example 1

### Identification of a Maize Having an Insert in the GDH2 Gene

A maize line having an insertion of a transposable element between position chr10:135303729-135303730 (RefGenV4) of the reference sequence in the GDH2 gene (Zm00001d025984) is isolated. The allele thus obtained is named D0425.

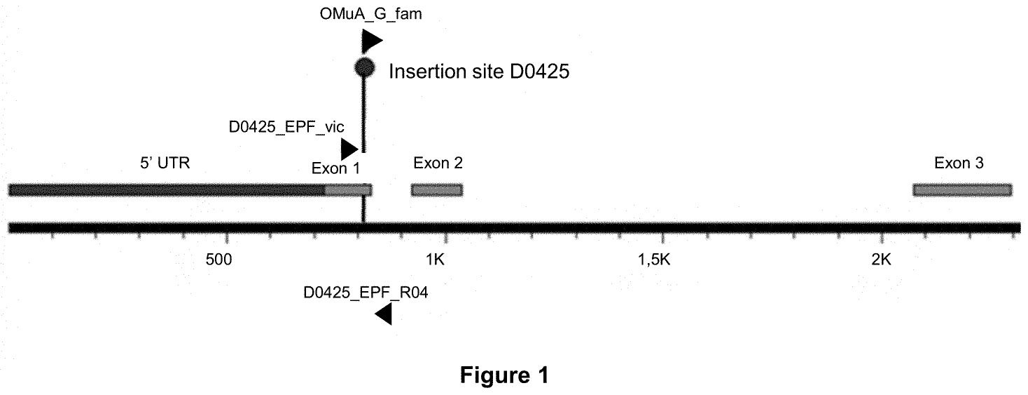

The insert of the transposable element is located in the end of the first exon (translated region) of the GDH2 gene (FIG. 1).

In order to determine if the insertion is in homozygous or heterozygous form, three primers were defined according to the PCR-based KASP technology: one allele-specific forward primer of the GDH2 sequence (named D0425_EPF_F04_vic: ATCGAAGCTGCTCGGCCTC (SEQ ID NO: 22)) with a proprietary tail sequence corresponding with VIC dye, one allele-specific forward primer of the endogenous transposable element (named OMuA_G_fam: CTTCGTCCATAATGGCAATTATCTCG (SEQ ID NO: 23)) with a proprietary tail sequence corresponding with FAM dye and a third common allele-specific reverse primer of the GDH2 gene (named D0425_EPF_R04: AGACGCCACAAGCAACACG (SEQ ID NO: 24)).

These three primers may be used simultaneously in a PCR amplification experiment (Kaspar protocol LGC Genomics) starting with genomic DNA (hybridization temperature=57° C.). End point fluorescence read, and clusters analysis of the samples reveal:
Vic fluorescence for homozygous WT plants
Fam fluorescence for homozygous mutant plants;
Both vic and fam fluorescence for the heterozygous plants.

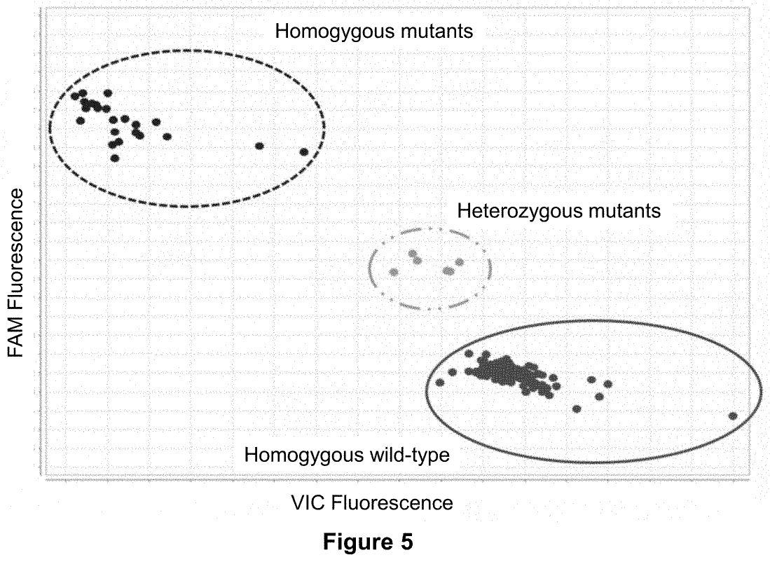

The results, presented in FIG. 5 show the presence of homozygous and heterozygous mutants.

### Example 2

### Crossing to Obtain the Homozygous And Heterozygous Lines Tested in 2017 Trials

Introgression lines carrying or not the mutation were constructed so as to obtain mutants and a control differing only by the presence of the mutation. The introgression lines obtained were then crossed with each other in order to evaluate homozygous, heterozygous and wild type hybrids in a trial on summer 2017.

### Example 3

### In Gel Activity of the WT and GDH2 Homozygous and Heterozygous Lines

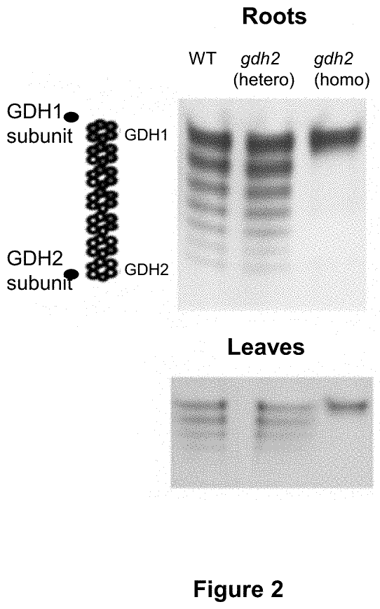

Protein extracts of the roots and leaves of the L1 wild type (WT) and L1 GDH2 homozygous and heterozygous mutants were subjected to native PAGE followed by NAD-GDH in-gel activity staining (Restivo et al. 2004) (FIG. 2). The different GDH1 and GDH2 subunit combinations of the seven isoenzymes detected in the L1 WT are indicated on the left side of the panel. Seven bands of NAD-GDH in-gel activity were detected in the L1 WT composed of different combinations of GDH1 and GDH2 subunits, whereas only one band of GDH1 activity was directed in the L1 GDH2 homozygous mutant containing GDH1 homohexamer.

### Example 4

### Phenotype Analysis of the D0425 Mutant in a Hybrid Context for the Improvement of Yield in Optimal Condition on Summer 2017

Homozygous mutant hybrids, heterozygous mutant hybrids and wild type hybrids as a control were all evaluated in a trial on summer 2017. The experiment was carried out according to the following protocol:

1 location (St PAUL Les Romans/Drome/France)

6 replicates

optimal condition for water and nitrogen requirements (OPT)

The measured traits were:

Grain yield 15% (GY15%): shelled grain weight per plot adjusted to 15% grain moisture and converted to quintals per hectare.

Kernel numbers per square meters (K/m²): grain numbers per square meters calculated from grain yield estimation and thousand kernels weight

Thousand kernels weight (TKW): Weight of 1000 kernels randomly selected from the total kernels and adjusted to 15% moisture content.

Statistical analyses (ANOVA) were carried out to know if there was a difference between the different types of hybrids (homozygous for the mutation (mm), heterozygous for the mutation (m+) and wild-type control (wt)).

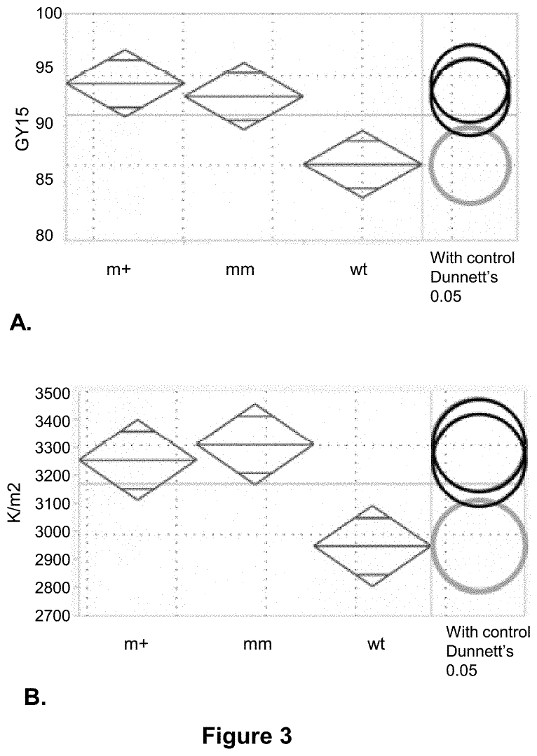

The results demonstrate that the insertion of a transposon into the GDH2 gene significantly (pvalue<5%) increases grain yield and kernel number per squares meters in optimal conditions (FIG. 3).

For grain yield, the analysis of variance is

TABLE 1
Analysis of variance for grain yield with reference to FIG. 3A

| | | Sum of | Mean | | |
|---|---|---|---|---|---|
| Source | DF | squares | square | F Ration | Prob > F |
| Allele | 2 | 178.45789 | 89.2289 | 7.6745 | 0.0051* |
| Error | 15 | 174.40032 | 11.6267 | | |
| C. Total | 17 | 352.85820 | | | |

For kernel number per surface unit, the analysis of variance is

TABLE 2
Analysis of variance for grain yield with reference to FIG. 3B

| | | Sum of | Mean | | |
|---|---|---|---|---|---|
| Source | DF | squares | square | F Ration | Prob > F |
| Allele | 2 | 461218.66 | 230609 | 8.4121 | 0.0035* |
| Error | 15 | 411208.41 | 27414 | | |
| C. Total | 17 | 872427.07 | | | |

The analysis of variance for both parameters shows that the measured difference of yield and kernel number between the wild-type and mutant plants is significant.

### Example 5

### Gene Editing Experiments

In Example 1, the transposon is positioned between bases 814 et 815 in the GDH2 gene sequence (SEQ ID NO: 2). Such gene interruption within this region can be reproduced with gene editing technologies.

The GDH2 gene sequence of Zea mays (SEQ ID NO: 2) was analyzed in silico to detect possible PAM corresponding to SpCas9 and FnCpf1 in the region of the insertion in the mutant from Example 1.

Two targets were found for SpCas9 and one for FnCpf1 and so three guide RNAs were designed. (SEQ ID NO: 15-16-17), respectively guide SpCas9-Target-90, guide SpCas9-Target-96, and guide FnCpf1-Target-91.

The proTaU6::SpCas9-Target-90::polyT cassette sequence and proZmUBI_intZmUBI::SpCAS9::terAtNOS cassette sequence were cloned via restriction enzyme reaction into a destination binary plasmid. The binary destination vector which contains a HMWG promoter driving a reporter gene to product a green fluorescent protein and an actin promoter (proOsActin) driving a bar gene which confers herbicide basta resistance is a derivative of the binary vector pMRT (WO2001018192A3). Maize cells are transformed by Agrobacterium tumefaciens according to Komari et al (1996). Maize cultivar A188 is transformed with these agrobacterial strains essentially as described by Ishida et al (1996).

proTaU6: SEQ ID NO: 18

proZmUBI_intZmUBI: SEQ ID NO: 19

terAtNos: SEQ ID NO: 20

polyT: SEQ ID NO: 21

In the same way, proTaU6::SpCas9-Target-96::polyT cassette sequence and proZmUBI_intZmUBI::SpCAS9::terAtNOS cassette sequence were cloned via restriction enzyme reaction and transformed into maize cells.

In the same way, proTaU6::FnCpf1-Target-91::polyT cassette sequence and proZmUBI_intZmUBI::FnCpft:terAtNOS cassette sequence were cloned via restriction enzyme reaction and transformed into maize cells.

### Example 6

### Root Amino-Acid Content in the GDH2 Mutants

The roots and shoots of homozygous mutant hybrids (mm), heterozygous mutant hybrids (m+) and wild type hybrids (WT) were sampled using plants having 6 fully developed leaves. Plants were grown on coarse sand in a controlled environment growth chamber (16 h light, 350-400 mmol photons.m-2·s-1, 26° C.; 8 h dark, 18° C.) and watered with a C solution containing 10 mM NO3- and 2 mM NH4+ (Cöic and Lesaint 1971). Amino acid extraction and quantification by GC-MS analysis were conducted as described by Cukier et al. (2018).

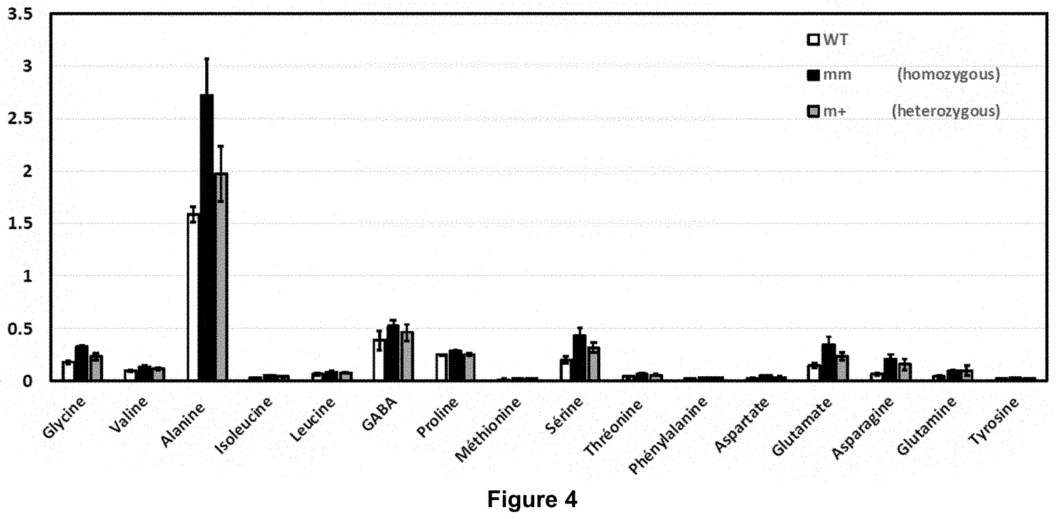

At 50-80% increase in the glutamate content and of all the amino acids derived from glutamate (Alanine, GABA, Asparagine, Glutamine, Serine, Glycine) was observed in the roots of maize hybrids carrying a homozygous mutation (mm) for the gene encoding Gdh2 (FIG. 4).

Such an increase was less marked in the heterozygous hybrid mutants (m+) suggesting a dose-dependent effect of the mutation. Such an increase was not observed in shoots likely because the enzyme activity is at least five times higher in roots compared to the shoots (FIG. 2), an organ in which gdh mutations induce more important physiological modifications in comparison those observed in the shoots (Fontaine et al., 2012). The increase in glutamate and derived amino acid is in line with the finding that GDH deaminates glutamate (Labboun et al., 2009), thus leading to an accumulation of amino acids when the enzyme is less active in the mutant. No significant differences were observed in the shoot amino acid content of the shoots of the two types of mutants compared to the wild type (data not shown).

### REFERENCES

Coïc Y, Lesaint C (1971) Comment assurer une bonne nutrition en eau et en ions minéraux en horticulture. Hortic Française 8: 11-14

Cukier, C., Lea, P. J., Cañas, R., Marmagne, A., Limami, A. M., Hirel, B. 2018 Labeling maize (Zea mays L.) leaves with 15NH₄⁺ and monitoring nitrogen incorporation into amino acids by GC/MS analysis. Curr. Prot. Plant Biol. doi.org/10.1002/cppb.20073.

Dubois F, Tercé-Laforgue T, Gonzalez-Moro M B, Estavillo M B, Sangwan R,

Gallais A, Hirel B (2003) Glutamate dehydrogenase in plants: is there a new story for an old enzyme? Plant Physiol Biochem. 41: 565-576.

Fontaine J X, Saladino F, Agrimonti C, Bedu M, Tercé-Laforgue T, Tétu T, Hirel B, Restivo F M, Dubois F (2006) Control of the Synthesis and of the Subcellular Targeting of the Two GDH Genes Products in Leaves and Stems of Nicotiana plumbaginifolia and Arabidopsis thaliana. Plant Cell Physiol. 47: 410-418.

Fontaine J X, Tercé-Laforgue T, Armengaud P, Clément G, Renou J P, Pelletier S, Catterou M, Azzopardi M, Gibon Y, Lea P J, Hirel B, Dubois F (2012) Characterization of a NADH-dependent glutamate dehydrogenase mutant of Arabidopsis demonstrates the key role of this enzyme in root carbon and nitrogen metabolism. Plant Cell 24: 4044-4065.

Fontaine J X, Tercé-Laforgue T, Bouton S, Pageau K, Lea P J, Dubois F, Hirel B (2013) Further insights into the isoenzyme composition and activity of glutamate dehydrogenase in Arabidopsis Thaliana. Plant Signal. Behay. 8: 3, e233291-5.

Grabowska A, Zdunek-Zastocka E, Kutryn E, Kwinta J (2017) Molecular cloning and functional analysis of the second gene encoding glutamate dehydrogenase in triticale. Acta Physiol. Plant. 39:24

Hirel B, Martin A, Tercé-Laforgue T, Gonzalez-Moro M B, Estavillo J M (2005) Physiology of maize I: A comprehensive and integrated view of nitrogen metabolism in a C4 plant. Physiol. Plant. 124: 167-177.

Hirel B, Le Gouis J, Ney B, Gallais A (2007) The challenge of improving nitrogen use efficiency in crop plants: towards a more central role for genetic variability and quantitative genetics within integrated approaches. J. Exp. Bot. 58: 2369-2387.

Labboun S, Tercé-Laforgue T, Roscher A, Bedu M, Restivo F M, Velanis C N, Skopelitis D S, Moshou P N, Roubelakis-Angelakis K A, Suzuki A. Hirel B (2009) Resolving the role of plant glutamate dehydrogenase: I. In vivo real time nuclear magnetic resonance spectroscopy experiments. Plant Cell Physiol 50: 1761-1773.

Lightfoot D A, Mungur R, Ameziane R, Nolte S, Long L, Bernhard K, Colter A, Jones K, Iqbal M J, Varsa E, Young B (2007) Improved drought tolerance of transgenic Zea mays plants that express the glutamate dehydrogenase gene (gdhA) of E. coli. Euphytica 156: 103-116.

Loulakakis K A, Roubelakis-Angelakis K A (1992) Ammonium-induced increase in NADH-glutamate dehydrogenase activity is caused by de novo synthesis of the α-subunit. Planta 187: 322-327.

Loulakakis K A, and Roubelakis-Angelakis K A (1996) The seven NAD(H)-glutamate dehydrogenase isoenzymes exhibit similar anabolic activities. Physiol. Plant. 96: 29-35.

Melo-Oliveira R, Oliveira I C, Coruzzi G M (1996) Arabidopsis mutant analysis and gene regulation define a non-redundant role for glutamate dehydrogenase in nitrogen assimilation. Proc. Natl. Acad, Sci. USA 96: 4718-4723.

Pavesi A, Ficarelli A, Tassi F, Restivo F M (2000) Cloning of two glutamate dehydrogenase cDNAs from Asparagus officinalis: sequence analysis and evolutionary implications. Genome 4: 306-316.

Purnell M P, Skopelitis D S, Roubelakis-Angelakis K A, Botella J R (2005) Modulation of higher-plant NADH-dependent glutamate dehydrogenase activity in transgenic tobacco via alteration of beta subunit levels. Planta 222: 167-180.

Restivo F M (2004) Molecular cloning of glutamate dehydrogenase genes of Nicotiana plumbaginifolia: structure and regulation of their expression by physiological and stress conditions. Plant Sci. 166: 971-982.

Skopelitis D S, Paranychiankis N V, Paschalidis K A, Plianokis E D, Delis I D, Yakoumakis D I, Kouvarakis A, Papadakis E D, Stephanou E G, Roubelakis-Angelakis K A (2006) Abiotic stress generates ROS that signal expression of anionic glutamate dehydrogenase to form glutamate for proline synthesis in tobacco and grapevine. Plant Cell 18: 2767-2781.

Tercé-Laforgue T, Bedu M, Dargel-Graffin C, Dubois F, Gibon Y, Restivo F M, Hirel B, (2013) Resolving the role of plant glutamate dehydrogenase: II. Physiological Characterization of plants overexpressing individually or simultaneously the two enzyme subunits. Plant. Cell. Physiol. 54: 1634-1647.

Tercé-Laforgue T, Clément G, Marchi L, Restivo F M, Lea P J, Hirel B (2015). Resolving the role of plant NAD-glutamate dehydrogenase: Ill. Overexpressing individually or simultaneously the two enzyme subunits under salt stress induces changes in the leaf metabolic profile and increases plant biomass production. Plant Cell Physiol. 56: 1918-1929.

Turano F J, Dashner R, Upadhyaya A, Caldwell C R (1996) Purification of mitochondrial glutamate dehydrogenase from dark-grown soybean seedlings. Plant Physiol. 112: 1357-1364.

Zhou X, Lin J, Zhou Y, yang Y, Liu H, Zhang C, Tang D, Zhao X, Zhu Y, Liu X (2015) Overexpressing a fungal CeGDH gene improves nitrogen utilization and growth in rice. Crop Sci 55: 811-820.

1.-15. (canceled)

16. A cereal comprising at least one cell which presents an inhibition of GDH2 activity.

17. The cereal of claim 16, wherein the inhibition of GDH2 activity is due to a mutation in the gdh2 gene.

18. The cereal of claim 16, wherein the mutation in the gdh2 gene is obtained by one of the following methods:
a. a physical treatment, wherein the physical treatment comprises applying an electromagnetic radiation or a particle radiation to the cereal
b. a chemical treatment, wherein the chemical treatment comprises exposing the seeds, gametes or plant parts of the cereal to a chemical, or
c. an engineering biological system, wherein the engineering biological system comprises Gene editing, base editing or Genetic Modification.

19. The cereal of claim 17, wherein the sequence of the gdh2 gene encodes the GDH2 enzyme depicted by SEQ ID NO: 1.

20. The cereal of claim 16, which is maize.

21. The cereal of claim 20, wherein the inhibition of GDH2 activity is due to an insertion between nucleotides 814 and 815 of SEQ ID NO: 2.

22. The cereal of claim 20, wherein inhibition of GHD2 activity is due to introduction of a mutation in SEQ ID NO: 2 by gene editing.

23. The cereal of claim 16, wherein inhibition of GHD2 activity is due to the presence in the genome of said cereal of an antisense, or of an overexpression construct (leading to co-suppression), or of an RNAi construct.

24. A method for improving yield in a cereal, comprising inhibiting GDH2 activity in said cereal, wherein said cereal with inhibited GDH2 activity presents a higher yield than a cereal not having inhibited GDH2 activity.

25. The method of claim 24, wherein said cereal is maize.

26. The method of claim 24, wherein the inhibition of GDH2 activity is obtained by:
a. an insertional mutagenesis or the introduction of at least one point mutation,
b. the expression of an antisense or RNAi construct or the overexpression of a sense construct to cause co-suppression wherein the constructs are integrated in the cereal's genome, or
c. the removal of part of the gdh2 gene or of the entire gene

27. A method for increasing cereal yield, comprising sowing cereal seeds, wherein said cereal seeds grow into plants that exhibit inhibited GDH2 activity, and wherein the yield of the cereals is increased as compared to the yield obtained from cereals not exhibiting inhibited GDH2 activity.

28. A method for identifying the cereal of claim 16 comprising:
(a) screening a population of cereals, and
(b) identifying the cereals presenting inhibition of GDH2 activity.

29. The cereal of claim 17, which is maize.

30. The cereal of claim 19, which is maize.

31. The cereal of claim 19, wherein inhibition of GHD2 activity is due to the presence in the genome of said cereal of an antisense, or of an overexpression construct (leading to co-suppression), or of an RNAi construct.

32. The cereal of claim 20, wherein inhibition of GHD2 activity is due to the presence in the genome of said cereal of an antisense, or of an overexpression construct (leading to co-suppression), or of an RNAi construct.

33. The cereal of claim 16, wherein all cells present an inhibition of the GDH2 activity.

34. The cereal of claim 33, which is maize.


SEQUENCE LISTING (23 sequences)

SEQ ID NO: 1 - protein, 411 aa, Zea mays
MNALAATTRNFRRASKLLGLDSKLEQSLLIPFREIKVECTIPKDDGSLATFVGFRVQHDNARGPMKGGIRYHNEVDPDEVNALAQLMTWKTAVAAVPYGGAKGGIGCSPGELSRSELERLTRVFTQKIHDLIGTHTDVPAPDMGTNAQTMAWMLDEYSKFHGHSPAVVTGKPIDLGGSLGRDAATGRGVMYATEALLAEYGKCISGSTFVIQGFGNVGSWAARLIHEKGGKIIAIGDVTGSIRNTAGIDIPALVKHRNEGHAMKDFDGAEVLDSTELLVHDCDVLVPCALGGVLNKDNAPDVKAKFVIEAANHPTDPEADEILAKKGVVVLPDIYANSGGVVVSYFEWVQNIQGFMWDEEKVNDELEKYMSSAFQHMKAMCKSLDCDLRMGAFTLGVNRVARATLLRGWEA

SEQ ID NO: 2 - DNA, 1236 bp, Zea mays
atgaacgcgctcgccgccaccacccgcaacttccggcgggcatcgaagctgctcggcctcgactccaagctcgagcagagcctgctcatcccgttccgggagatcaaggtggaatgcaccatccccaaagacgacggcagcttggcgacgttcgtggggttccgcgtgcagcatgataacgcccgcgggccgatgaaaggcggcatccgctatcacaatgaggttgatccagatgaagtaaatgcccttgctcaactcatgacatggaagacagctgttgctgcagtcccatatggtggagcaaagggagggatcgggtgctctcctggtgaactaagtagaagtgagttggagcggttgacacgagtatttacccagaaaattcatgatcttatcggaacgcatacagatgtccctgctcctgacatggggaccaatgcacaaaccatggcatggatgctggatgagtactcgaaatttcatggtcactccccagcagtcgtcactgggaagccaatagatcttggcggatcactgggcagggatgcagcaacagggcgaggcgtaatgtatgctaccgaggccctactcgctgaatatggaaaatgcatttctggatcaacttttgtgatccaaggttttggcaatgttggttcatgggctgctcgacttattcacgagaaaggtggtaagattattgcaattggggatgtgacaggttcaattagaaacacggctggtatagacatacctgctttggtgaagcataggaatgaaggtcatgcgatgaaagacttcgatggtgcggaagttttggattcaaccgagttgttagtgcatgactgtgatgttcttgtcccctgcgccttaggtggagttcttaacaaggataatgcaccagacgtgaaggccaaatttgtaatcgaagctgctaaccatccaactgatccagaggcagacgagattctcgccaagaagggggtagtagtattacctgatatatatgctaattcaggtggcgtggtcgttagctactttgaatgggtgcagaacattcaaggtttcatgtgggatgaggagaaagttaacgatgaactagaaaagtacatgagcagtgcttttcaacacatgaaagccatgtgcaaatctctggattgcgaccttaggatgggggccttcaccttaggagttaacagggttgctcgtgccacccttttgagaggctgggaggcatga

SEQ ID NO: 3 - protein, 411 aa, Sorghum bicolor
MNALAATSRNFRRASKLLGLDSKLEQSLLIPFREIKVECTIPKDDGTLASFVGFRVQHDNARGPMKGGIRYHPEVDPDEVNALAQLMTWKTAVAAVPYGGAKGGIGCSPGELSRSELERLTRVFTQKIHDLIGTHTDVPAPDMGTNAQTMAWMLDEYSKFHGHSPAVVTGKPIDLGGSLGRDAATGRGVMYATEALLAEYGKCISGSTFVIQGFGNVGSWAARLIHEKGGKIIAIGDVTGSIKNMSGIDIPALMKHKNEGHAMKDFHGAEVMDSTELLVHECDVLVPCALGGVLNKDNAPSVKAKFIVEAANHPTDPEADEILAKKGVVVLPDIYANSGGVIVSYFEWVQNIQGFMWDEEKVNNELEKYMSSAFQHIKAMCKSLDCNLRMGAFTLGVNRVARATLLRGWEA

SEQ ID NO: 4 - protein, 411 aa, Oryza sativa
MNALAATSRNFRQAARLLGLDSKLQKSLLIPLREIKVECTIPKDDGTLATFVGFRVQHDNSRGPMKGGIRYHPEVDPDEVNALAQLMTWKTAVAAVPYGGAKGGIGCTPGELSRSELERLTRVFTQKIHDLIGINTDVPAPDMGTNAQTMAWILDEYSKFHGHSPAVVTGKPIDLGGSLGRDAATGRGVMYATEALLTEYSESISGSTFVIQGLGNVGSWAAKLIHQKGGKIVAVGDVTGAIRNKSGIDIPALLKHRSEGGSLEDFYGAEVMDAAELLVHECDVLVPCALGGVLNRENAAEVKARFIIEGANHPTDTEADEILAKKGVIVLPDIYANSGGVVVSYFEWVQNIQGFMWDEEKVNRELQKYMKNAFQNIKDMCKSQNCNLRMGAFTLGVNRVAKATLLRGWEA

SEQ ID NO: 5 - protein, 262 aa, Hordeum vulgare
MAWIFDEYSKFHGHSPAVVTGKPIDLGGSLGRDAATGRGVMYATEALLAEYGKSISGSTFVIQGFGNVGSWAAQLIHEKGGKVIALGDVSGTIRNKAGIDVPALMKHRNEGGQLKDFHGAEVMDSSELLVHECDVLLPCALGGVLNKDNAPDVKAKFIIEAANHPTDPEADEILTKKGVVVLPDIYANAGGVIVSYFEWVQNIQGFMWEEEKVNMELHKYMNSAFQHIKAMCKSQDCSLRMGAFTLGVNRVARATILRGWEA

SEQ ID NO: 6 - protein, 411 aa, Triticum aestivum
MNALAATSRNFRQAARLLGLDSKLEKSLLIPFREIKVECTIPKDDGTLASFVGFRVQHDNARGPMKGGIRYHPEVDPDEVNALAQLMTWKTAVAAVPYGGAKGGIGCSPGDLSRSELERLTRVFTQKIHDLIGTHTDIPAPDMGTNSQTMAWIFDEYSKFHGHSPAVVTGKPIDLGGSLGRDAATGRGVMYATEALLAEYGKSISGSTFVIQGFGNVGSWAAQLIHEKGGKVIALGDVSGTIRNKAGIDVPALMKHRNEGGQLKDFHGAEVMDASELLVHECDVLLPCALGGVLNRENAPDVKAKFIIEAANHPTDPEADEILTKKGVVVLPDIYANAGGVIVSYFEWVQNIQGFMWEEEKVNMELHKYMNSAFQNIKAMCKSQDCNLRMGAFTLGVNRVARATILRGWEA

SEQ ID NO: 7 - protein, 411 aa, Triticum aestivum
MNALAATSRNFRQAARLLGLDSKLEKSLLIPFREIKVECTIPKDDGTLASFVGFRVQHDNARGPMKGGIRYHPEVDPDEVNALAQLMTWKTAVAAVPYGGAKGGIGCSPGDLSRSELERLTRVFTQKIHDLIGTHTDIPAPDMGTNSQTMAWIFDEYSKFHGHSPAVVTGKPIDLGGSLGRDAATGRGVMYATEALLAEYGKSISGSTFVIQGFGNVGSWAAQLIHEKGGKVIALGDVSGTIRNKAGIDVPALMKHRNEGGQLKDFHGAEVMDASELLVHECDVLLPCALGGVLNRENAPEIKAKFIIEAANHPTDPEADEILTKKGVVVLPDIYANAGGVIVSYFEWVQNIQGFMWEEEKVNMELHKYMNSAFQNIKAMCKSQDCNLRMGAFTLGVNRVARATILRGWEA

SEQ ID NO: 8 - protein, 411 aa, Triticum aestivum; identical to SEQ ID NO: 6

SEQ ID NO: 9 - DNA, 1236 bp, Sorghum bicolor
atgaacgcgctcgccgccaccagccgcaacttccggcgggcctcgaagctgctcggcctcgactccaagctcgagcagagtctgctcatcccgttccgggagatcaaggtggaatgcaccatccccaaagacgacggcaccttggcgtcgttcgtcggcttccgcgtgcagcatgacaacgcccgcgggccgatgaaaggcggcatccgctaccatcctgaggttgatccagatgaagtaaacgcccttgctcaactcatgacatggaagacagctgttgcagcagttccatatggtggagcaaagggagggatcggatgctctcctggtgaactaagtagaagtgagttggagcggttgacgcgagtatttacccagaaaattcatgatcttatcggaacgcatacagatgtcccagctcctgacatggggaccaatgcacagaccatggcatggatgttggatgagtactcgaaatttcatggtcactcaccagcagtcgtcacaggcaagccaattgatcttggtggatcactgggtagggatgcagcaacagggcgaggtgtcatgtatgcgaccgaggctctacttgctgaatatggaaaatgtatttctggatcaacttttgtgattcaaggttttggcaatgttggctcatgggctgcacgacttatccatgagaaaggtggtaagattattgcaattggagatgtgacgggttcaatcaaaaacatgtcgggtatagacatacctgctttgatgaagcataagaatgaaggtcatgccatgaaagactttcatggtgcagaagtcatggattcgactgagttgctagtgcacgaatgtgatgttcttgtcccttgtgccttaggtggagttcttaacaaggataatgcaccaagcgtgaaggccaaatttatagttgaagctgctaaccatccaactgatccagaggcagacgagattctcgccaagaagggagtggtcgtattacctgatatctatgctaattcaggtggtgtgatcgttagctactttgagtgggttcagaacattcaaggtttcatgtgggatgaagagaaagtgaacaatgaactagagaagtacatgagcagtgcttttcaacacatcaaggccatgtgcaagagtctggattgcaaccttaggatgggggcattcaccttaggagttaacagggttgcccgtgccaccctcttgagaggctgggaggcatga

SEQ ID NO: 10 - DNA, 1236 bp, Oryza sativa
atgaacgcgctagccgccaccagccgcaacttccggcaggcggcgaggctactcggcctcgactccaaactccagaagagccttctgatccctctccgcgagatcaaggttgaatgcaccatccccaaagacgatggcaccttggcgacgtttgttgggttcagagtgcagcatgacaattcccgtggcccgatgaaaggcggcatcaggtatcaccctgaggttgacccagatgaagtgaatgcacttgctcagctgatgacatggaagactgctgttgctgctgtgccttatggtggagcaaagggaggcattgggtgcactcctggtgaactgagtagaagtgaacttgagcgtttaacgcgcgtgtttacccagaaaattcatgacctcattgggatcaatacagatgttccagcccctgacatgggaacaaatgcacagaccatggcatggatactagatgaatactcaaagttccatggccactccccagctgttgtcacgggaaagccaattgatcttggaggatccttaggaagagatgctgcgactggccgcggtgttatgtatgctactgaggctttattgactgaatacagcgagtcaatttcaggatcaacctttgttatccagggtttggggaatgttggttcttgggcagccaagctcatccatcagaaaggtggcaagatagttgcagttggagatgtaacaggtgcaatcagaaataaatccgggatagacatacctgctctcttgaagcacaggagcgagggtggatctttggaggacttctacggtgcagaagttatggacgctgcagagctgcttgtgcatgaatgtgatgttctcgtcccgtgcgccttgggtggtgttctcaacagagaaaatgcagctgaagtgaaggccagattcataattgagggggctaatcatcccactgatacagaagctgatgagatactcgcaaagaagggagtcattgtgctgccagatatctatgcaaattcaggcggcgtggtcgtcagctattttgagtgggttcagaacattcagggcttcatgtgggatgaagagaaggtgaacagggagctccaaaagtacatgaagaacgcttttcagaacatcaaggacatgtgcaaatcccaaaactgcaaccttaggatgggggcattcactttgggggttaaccgggttgcaaaggctactctgttgaggggatgggaggcatga

SEQ ID NO: 11 - DNA, 798 bp, Hordeum vulgare
agacagaccatggcatggatctttgacgagtactcaaaattccatggtcactccccagctgttgtcacagggaagcccatagatcttggtggatcattaggtagggatgctgccactgggcggggtgtaatgtatgctactgaggctctcctggctgaatacggaaagtctatttctggatcaacctttgttattcagggattcggtaatgttggttcatgggctgcacaactcatccatgagaaaggtggtaaggtaattgcacttggagatgtatcaggcacaatcagaaacaaagcagggatagacgtacctgctctgatgaagcacagaaatgagggtggtcagttgaaagactttcatggtgcggaagtcatggattcctcagagttgctggtgcatgaatgcgatgtcctcctcccatgcgccttaggtggagttcttaacaaagataatgcacctgatgtgaaggccaaatttataatcgaagctgctaatcatccaactgatccagaagctgatgagatccttaccaagaagggggtggtcgtgttacctgatatctatgctaatgctggtggtgtgatcgttagttattttgagtgggttcagaacattcaaggcttcatgtgggaagaagaaaaggtgaacatggagctccacaagtatatgaacagcgcattccagcacatcaaggccatgtgcaagtctcaagattgcagccttaggatgggagcattcaccttgggagtgaaccgggttgcacgcgccaccatcttgaggggctgggaggcatga

SEQ ID NO: 12 - DNA, 1236 bp, Triticum aestivum
atgaacgcgctcgctgcgaccagccgcaacttccgccaggccgcccggctgctcggcctcgactccaagctcgagaagagcctcctcatccccttccgcgagatcaaggtcgaatgcaccatccccaaggacgacggcacgctggcctccttcgtcggcttccgcgtgcagcacgacaatgcccgcgggcccatgaaaggtggcatccgctaccaccccgaggttgatccagatgaagtaaatgcacttgctcagctgatgacatggaagactgccgttgcggcagtaccatatggtggagcaaagggaggaatagggtgctctcccggtgatttaagtaggagtgagttggagcgtctgacgcgtgtatttactcagaaaattcatgaccttatcggaactcataccgatattccagctccagacatgggaactaattcacagaccatggcatggatctttgacgagtactcaaaattccatggtcactccccagctgttgtcacagggaagcccatagatcttggtggatcattaggtagggatgctgccactgggcggggtgtaatgtacgctactgaggctctcctggccgaatatgggaagtctatttctggatcaacctttgttattcagggattcggtaatgttggttcatgggctgcacaactcatccatgagaaaggtggtaaggtaattgcacttggagatgtatcaggcacaatcagaaacaaagcagggatagacgtacctgctctgatgaagcacagaaatgagggtggtcagttgaaagactttcatggcgctgaagtcatggatgcctcagagttgctagtgcatgaatgcgatgtcctcctcccatgtgccttaggcggagttcttaacagggaaaatgcccctgatgttaaggccaaatttataatcgaagctgctaatcatccaaccgatccagaagctgatgagattcttaccaagaagggagtggtcgtgttgcctgatatctatgctaatgctggtggtgtgatcgttagctattttgagtgggttcagaacattcaaggattcatgtgggaagaagaaaaggtgaacatggagctccataagtacatgaacagcgcgttccagaacatcaaggccatgtgcaagtctcaagattgcaaccttaggatgggagcattcaccttgggagtgaaccgggttgctcgcgccaccatcttgaggggctgggaggcatga

SEQ ID NO: 13 - DNA, 1236 bp, Triticum aestivum
atgaacgcgctcgccgcgaccagccgcaacttccgccaggccgcccgcctgctcggcctcgactccaagctcgagaagagcctcctcatccccttccgcgagatcaaggtcgaatgcaccatccccaaggacgacggcacgctggcctccttcgtgggcttccgcgtgcagcacgacaatgcccgcgggcccatgaaaggtggcatccgctaccaccccgaggttgatccagatgaagtaaatgcacttgctcagctgatgacatggaagactgccgttgcggcagtaccatatggtggagcaaagggaggaatagggtgctctcccggtgatttaagtaggagtgagttggagcgtctgacgcgtgtatttactcagaaaattcatgaccttatcggaactcataccgatattccagctccagacatgggaactaattcacagaccatggcatggatctttgacgagtactcaaaattccatggtcactccccagctgttgtcacagggaagcccatagatcttggtggatcattaggtagggatgcggccactgggcggggtgtaatgtacgctactgaggctctcctggccgaatatgggaagtctatttctggatcaacctttgttattcagggattcggtaatgttggttcatgggctgcacaactcatccatgagaaaggtggtaaggtaattgcacttggagatgtatcaggcacaatcagaaacaaagcaggaatagacgtacctgctctgatgaagcacagaaatgagggtggtcagttgaaagactttcatggcgctgaagtcatggatgcctcagagttgctagtgcatgaatgcgatgtcctcctcccatgtgccttaggcggggttcttaacagggaaaatgcccctgaaattaaggccaaatttataatcgaagctgctaatcatccaaccgatccagaagctgatgagattcttaccaagaagggagtggtcgtgttacctgatatctatgctaatgctgggggtgtgatcgttagctattttgagtgggttcagaacattcaaggattcatgtgggaagaagaaaaggtgaacatggagctccataagtacatgaacagcgcgttccagaacatcaaggccatgtgcaagtctcaagattgcaaccttaggatgggagcattcaccttgggagtgaaccgggttgctcgcgccaccatcttgaggggttgggaggcatga

SEQ ID NO: 14 - DNA, 1236 bp, Triticum aestivum
atgaacgcgctcgccgcgaccagccgcaacttccgccaggccgcccggctgctcggcctcgactccaagctcgagaagagcctcctcatccccttccgcgagatcaaggtcgaatgcaccatccccaaggacgacggcacgctggcctccttcgtgggcttccgcgtgcagcacgacaatgcccgcgggcccatgaaaggtggcatccgctaccaccccgaggttgatccagatgaagtaaacgcacttgctcaactgatgacatggaaaactgccgttgcggctgtaccatatggtggagcaaagggaggaatagggtgctctcctggtgatttaagtaggagtgagttggagcgtctgacgcgtgtatttactcagaaaattcatgaccttattggaactcataccgatattccagctccagacatgggaactaattcacagaccatggcatggatctttgacgagtactcaaaattccatggtcactccccagctgttgtcacagggaagcccattgatcttggtggatcattaggtagggatgctgccactgggcggggtgtaatgtacgctactgaggctctcctggccgaatatgggaagtctatttctggatcgacctttgttattcagggattcggtaatgttggttcatgggctgcacaactcatccatgagaaaggtggtaaggtaattgcacttggagatgtatcaggcacaatcagaaacaaagcagggatagacgtacctgctctgatgaagcacagaaatgagggtggtcagttgaaagactttcatggcgctgaagtcatggatgcctcagagttgctagtgcatgaatgcgatgtcctcctcccatgtgccttaggcggagttcttaacagggaaaatgcccctgatgttaaggccaaatttataatcgaagctgctaatcatccaaccgatccagaagctgacgagattcttaccaagaagggagtggtcgtgttgcctgatatctatgctaatgctggtggtgtgatcgttagctattttgagtgggttcagaacattcaaggattcatgtgggaagaagaaaaggtgaacatggagctccataagtacatgaacagcgcgttccagaacatcaaggccatgtgcaagtctcaagattgcaaccttaggatgggagcattcaccttgggagtgaaccgggttgctcgcgccaccatcttgaggggctgggaggcatga

SEQ ID NO: 15 - DNA, 100 bp, Artificial Sequence
  guide SpCas9-Target-90
tctcccggaacgggatgagcgttttagagctagaaatagcaagttaaaataaggctagtccgttatcaacttgaaaaagtggcaccgagtcggtgctttt

SEQ ID NO: 16 - DNA, 100 bp, Artificial Sequence
  guide SpCas9-Target-96
catcccgttccgggagatcagttttagagctagaaatagcaagttaaaataaggctagtccgttatcaacttgaaaaagtggcaccgagtcggtgctttt

SEQ ID NO: 17 - DNA, 42 bp, Artificial Sequence
  guide FnCpf1-Target-91
aatttctactgttgtagatatctcccggaacgggatgagcag

SEQ ID NO: 18 - DNA, 363 bp, Artificial Sequence
  proTaU6
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttg

SEQ ID NO: 19 - DNA, 1992 bp, Artificial Sequence
  proZmUBI_intZmUBI
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 20 - DNA, 250 bp, Artificial Sequence
  terAtNos
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactag

SEQ ID NO: 21 - DNA, 19 bp, Artificial Sequence
  allele specific forward primer
atcgaagctgctcggcctc

SEQ ID NO: 22 - DNA, 26 bp, Artificial Sequence
  forward primer of the endogenous transposable element
cttcgtccataatggcaattatctcg

SEQ ID NO: 23 - DNA, 19 bp, Artificial Sequence
  allele-specific reverse primer of the GDH2 gene
agacgccacaagcaacacg